(19) United States
(12) Patent Application Publication
(10) Pub. No.: US 2023/0377743 A1
(43) Pub. Date: Nov. 23, 2023

(54) REMOTE VENTILATION DASHBOARD SYSTEM

(71) Applicant: Covidien LP, Mansfield, MA (US)

(72) Inventors: Jason GRIMES, Boulder, CO (US); Lakshmi SOWRIRAJAN, Boulder, CO (US); Clifford SCHORR, Boulder, CO (US); Rabia QASEEM, Boulder, CO (US); Gary MILNE, Boulder, CO (US); Timothy WILSEY, Boulder, CO (US); Corey CARSON, Boulder, CO (US)

(73) Assignee: Covidien LP, Mansfield, MA (US)

(21) Appl. No.: 18/248,271

(22) PCT Filed: Oct. 28, 2021
(86) PCT No.: PCT/US2021/057108

§ 371 Date: Apr. 7, 2023

Related U.S. Application Data
(60) Provisional application No. 63/109,234, filed on Nov. 3, 2020.

(51) Int. Cl. G16H 40/67

(52) U.S. Cl. CPC G16H 40/67

(57) ABSTRACT

Methods, systems, and devices for displaying physiological data in a user interface are described. The method may include identifying a first table and displaying a first row including the first set of values for the first physiological parameter and a second row including the second set of values for the second physiological parameter. The method may further include receiving, from a medical device associated with a patient, measured values associated with the first physiological parameter and the second physiological parameter. In some cases, the method may include identifying a first display range and a second display range based at least in part on the received measured values. The method may also include displaying a first visual indicator and a second visual based on whether the first display range and the second display range visually overlap between the first row and the second row.

### CROSS REFERENCE

The present application for patent claims the benefit of U.S. Provisional Patent Application No. 63/109,234 by Grimes et al., entitled “REMOTE VENTILATION DASHBOARD SYSTEM,” filed Nov. 3, 2020 which is expressly incorporated by reference herein.

### BACKGROUND

The following relates generally to displaying physiological data in a user interface, and more specifically to a remote ventilation dashboard system.

In a healthcare facility such as a hospital, physiological parameters of the patient (e.g., heart rate, respiratory rate, blood pressure) may be monitored by one or more medical devices. Clinicians may remotely monitor the patient by accessing the patient data at a central nurse station or on any web enabled device connected to the network (e.g., smartphone or tablet).

As multiple patients are monitored, data from medical devices (e.g., measurements obtained from medical devices) and data recorded by clinicians monitoring the patients (e.g., measurements from manually performed tests, patient condition information, etc.) may be collected over time. The increased amounts of data for each patient may make it difficult to for clinicians to discern relevant patient information or identify patients that may be at risk. Accordingly, when a clinician attempts to review the patient data in existing user interfaces, it may be difficult to quickly and efficiently identify data that is relevant or beneficial to the clinician at the time of reviewing. In other examples, when a clinician attempts to review the patient data, it may be difficult to identify patients that may be experiencing a significant physiological event. In that case, the physical and physiological state of the patient may be at risk or belated diagnosis or misdiagnosis of a patient may occur as a result of the unorganized data.

### SUMMARY

The described features generally relate to methods, systems, devices, or apparatuses that support a remote ventilation dashboard system. Aspects of the present disclosure describe techniques for receiving and displaying, via a user interface, measured physiological data from one or more remotely monitored patients in a way that facilitates quick and accurate identification of measured values compared to threshold values. In some examples, the described techniques provide a method for identifying whether two or more related physiological parameters are within predefined ratios with respect to each other. For example, a ventilation dashboard server may display physiological data in a user interface by identifying a table from a set of tables where the table defines a ratio between a first set of values for a first physiological parameter (e.g., fraction of inspired oxygen (FiO2)) and a second set of values for a second physiological parameter (e.g., positive end-expiratory pressure (PEEP)). The ventilation dashboard server may display a first row including the first set of values and a second row including the set of values. In some cases, the first row and the second row are adjacent and aligned with each other. The ventilation dashboard server may receive measured values associated with the first physiological parameter and the second physiological parameter from a medical device (e.g., a ventilator).

Based on the received measured values, the ventilation dashboard server may identify a first display range for the first set of values and a second display range for the second set of values. The ventilation dashboard server may display a first visual indicator in the first row and second row that highlights the first display range and second display range. The ventilation dashboard server may display a second visual indicator in the first row and second row that updates a characteristic of the first visual indicator (e.g., changes the color of the first visual indicator) for the first display range and second display range based on the display ranges overlapping each other.

A method for displaying physiological data in a user interface is described. The method may include identifying a first table from a plurality of tables, wherein the first table defines a ratio between a first set of values for a first physiological parameter and a second set of values for a second physiological parameter, displaying a first row including the first set of values for the first physiological parameter and a second row including the second set of values for the second physiological parameter, wherein the first row and the second row are adjacent and aligned with each other, receiving, from a medical device associated with a patient, a first measured value associated with the first physiological parameter and a second measured value associated with the second physiological parameter, identifying a first display range of the first set of values for the first physiological parameter based at least in part on the received first measured value, identifying a second display range of the second set of values for the second physiological parameter based at least in part on the received second measured value, displaying, in the first row and in the second row, a first visual indicator that highlights the first display range in the first row and the second display range in the second row, and displaying, in the first row and in the second row, a second visual indicator that updates a characteristic of the first visual indicator for the first display range and the second display range based at least in part on whether the first display range and the second display range visually overlap between the first row and the second row.

Some examples of the method disclosed herein may further include highlighting the first display range and the second display range a first color based at least in part on the first display range and the second display range overlapping. In some examples of the method disclosed herein may further include highlighting the first display range and the second display range a second color based at least in part on the first display range and the second display range being non-overlapping.

Some examples of the method disclosed herein may further include highlighting a subset of the first set of values in the first row that matches the first measured value and highlighting a subset of the second set of values in the second row that matches the second measured value. In some examples, the subset of the first set of values or the subset of the second set of values comprises duplicated values.

Some examples of the method disclosed herein may further include determining that the first measured value is a value between a first value and a second value of the first set of values in the first row and highlighting the first value and the second value of the first set of values in the first row based at least in part on determining that the first measured value is a value between the first value and the second value of the first set of values.

Some examples of the method disclosed herein may further include determining that the second measured value is a value between a first value and a second value of the second set of values in the second row and highlighting the first value and the second value of the second set of values in the second row based at least in part on determining that the second measured value is a value between the first value and the second value of the second set of values.

Some examples of the method disclosed herein may further include receiving a user selection of the first table from the plurality of tables. In some examples of the method disclosed herein may further include displaying, in the user interface, a tile configured to indicate whether the first display range and the second display range overlap based at least in part on the determination. In some examples, the first physiological parameter comprises FiO2 and the second physiological parameter comprises PEEP.

Some examples of the method described herein may further include operations, features, means, or instructions for identifying a first table from a plurality of tables, wherein the first table defines a ratio between a first set of values for a first physiological parameter and a second set of values for a second physiological parameter, displaying a first row including the first set of values for the first physiological parameter and a second row including the second set of values for the second physiological parameter, wherein the first row and the second row are adjacent and aligned with each other, receiving, from a medical device associated with a patient, a first measured value associated with the first physiological parameter and a second measured value associated with the second physiological parameter, identifying a first display range of the first set of values for the first physiological parameter based at least in part on the received first measured value, identifying a second display range of the second set of values for the second physiological parameter based at least in part on the received second measured value, displaying, in the first row and in the second row, a first visual indicator that highlights the first display range in the first row and the second display range in the second row, and displaying, in the first row and in the second row, a second visual indicator that updates a characteristic of the first visual indicator for the first display range and the second display range based at least in part on whether the first display range and the second display range visually overlap between the first row and the second row.

### BRIEF DESCRIPTION OF THE DRAWINGS

FIG. 1 illustrates an example of a wireless patient monitoring system in accordance with aspects of the present disclosure.

FIG. 2 illustrates an example of a system for displaying physiological data in a user interface that supports remote ventilation dashboard system in accordance with aspects of the present disclosure.

FIG. 3 illustrates an example of a ventilation dashboard that supports remote ventilation dashboard system in accordance with aspects of the present disclosure.

FIG. 4 illustrates an example of a plurality of tables that supports remote ventilation dashboard system in accordance with aspects of the present disclosure.

FIG. 5A illustrates an example of a table that supports remote ventilation dashboard system in accordance with aspects of the present disclosure.

FIG. 5B illustrates an example of a table that supports remote ventilation dashboard system in accordance with aspects of the present disclosure.

FIG. 5C illustrates an example of a table that supports remote ventilation dashboard system in accordance with aspects of the present disclosure.

FIG. 5D illustrates an example of a table that supports remote ventilation dashboard system in accordance with aspects of the present disclosure.

FIGS. 6 and 7 show block diagrams of ventilation dashboard servers that support remote ventilation dashboard system in accordance with aspects of the present disclosure.

FIG. 8 shows a block diagram of a ventilation dashboard manager that supports remote ventilation dashboard system in accordance with aspects of the present disclosure.

FIG. 9 shows a diagram of a system including a ventilation dashboard server that supports remote ventilation dashboard system in accordance with aspects of the present disclosure.

FIG. 10 shows a flowchart illustrating methods that support remote ventilation dashboard system in accordance with aspects of the present disclosure.

### DETAILED DESCRIPTION

In some patient monitoring systems, medical devices may measure one or more physiological parameters of a patient and communicate the measurements associated with the physiological parameters to a central server or some other downstream device or system. The medical device may be an example of a ventilator or similar device related to measuring respiratory parameters of a patient. In some cases, a clinician may be concerned with how two or more measured physiological parameters relate to each other rather than (or in addition to) the absolute measurement of those parameters. In such cases, a clinician may be concerned with how a ratio of parameters compares with a predefined ratio of these parameters to assess whether a patient is experiencing an adverse physiological event. For example, with respect to the fraction of inspired oxygen (FiO2) and positive end-expiratory pressure (PEEP), the ratio of these two parameters may indicate a physiological event, whereas the individual measurements of these two values, by themselves, may be less indicative of a physiological event. Existing techniques for collecting and displaying such parameters may be limited to displaying the individual values of measured parameters (e.g., the current measured value for FiO2 and PEEP) in a display, and a clinician may have to manually assess whether the two displayed values, when compared to each other (e.g., a ratio or other comparison), falls within a predefined range. Such user interfaces may require manual look up or comparison between the two values and a predefined table by the clinician, which may increase the time and difficulty associated with assessing a patient's measured parameters. However, as described in more detail below, by configuring a remote ventilation dashboard in accordance with aspects of the present disclosure, a clinician monitoring the patient may be able to identify whether a combination of received measured values is within a predefined ratio threshold (or similar comparative metrics), discern an alarm of interest, and tend to the patient more quickly and accurately.

In accordance with various aspects described herein, medical data and notifications associated with the received measured values may be displayed via a patient tile screen on the remote ventilation dashboard, thereby increasing the accessibility to patient data to determine whether a ratio of measured parameters is within a threshold. For example, a ventilation dashboard server may generate a table by displaying a first row that includes a first set of values for a first physiological parameter and displaying a second row that includes a second set of values for a second physiological parameter. The ventilation dashboard server may receive measured values associated with the first and second physiological parameters from medical devices associated with the patient. In some cases, the first physiological parameter may include FiO2 and the second physiological parameter may include PEEP. However, additional or alternative physiological parameters may be analyzed and displayed in accordance with aspects of the present disclosure.

The ventilation dashboard server may identify display ranges for the first row and the second row based on the received measured values and display visual indicators within the table that highlights the display ranges. For example, the ventilation dashboard server may highlight the received measured value within the table or highlight a range of values that includes the received measured value. In some cases, the ventilation dashboard server may update a color of the display ranges based on whether the display ranges overlap. If the display ranges overlap, the display ranges may be a first color (e.g., green), and the patient tile on the remote ventilation dashboard may be the first color, thereby indicating that the patient's measured values are within the threshold. If the display ranges are non-overlapping, the display ranges may be a second color (e.g., red), and the patient tile on the remote ventilation dashboard may be the second color, thereby indicating that the patient's measured values are outside the threshold and the patient may be at risk. As described in more detail with respect to the figures, a user interface that includes adjacent tables or rows of parameter values that dynamically highlight ranges based on received medical values, and that update a color of the highlighting (or other visual or audio indicator change) based on whether the highlighted ranges are overlapping, may quickly indicate whether measured physiological parameters fall within predefined ratio limits with respect to each other. Such user interfaces and techniques for displaying medical data provide additional insight and remove ambiguity as compared to user interfaces that merely display the measured values of two or more physiological values, thereby requiring the clinician to manually compare the two values to each other and assess whether the comparison of the two values falls within a predefined ratio (or similar metric).

The individual indicator (e.g., the highlighted table and patient tile) may be unique to the individual patient and may be configured independently of the individual indicators for other patients. In some cases, displaying an indication of whether the display ranges overlap may provide access to real-time patient data anywhere the user may have access to the network associated with the ventilation dashboard server. In such cases, the remote ventilation dashboard may provide remote alarm visualization to reduce a number of overlooked patient issues. In some cases, the patient tile screen may allow the clinician to remotely view ventilation alarms and quickly access patient data, thereby allowing clinicians to remotely view real-time ventilator settings, parameters, and waveforms to keep clinicians informed and able to make informed treatment decisions.

Aspects of the disclosure are initially described in the context of a wireless patient monitoring system. Aspects of the disclosure are further illustrated by and described with reference to apparatus diagrams, system diagrams, tables, and flowcharts that relate to a remote ventilation dashboard system.

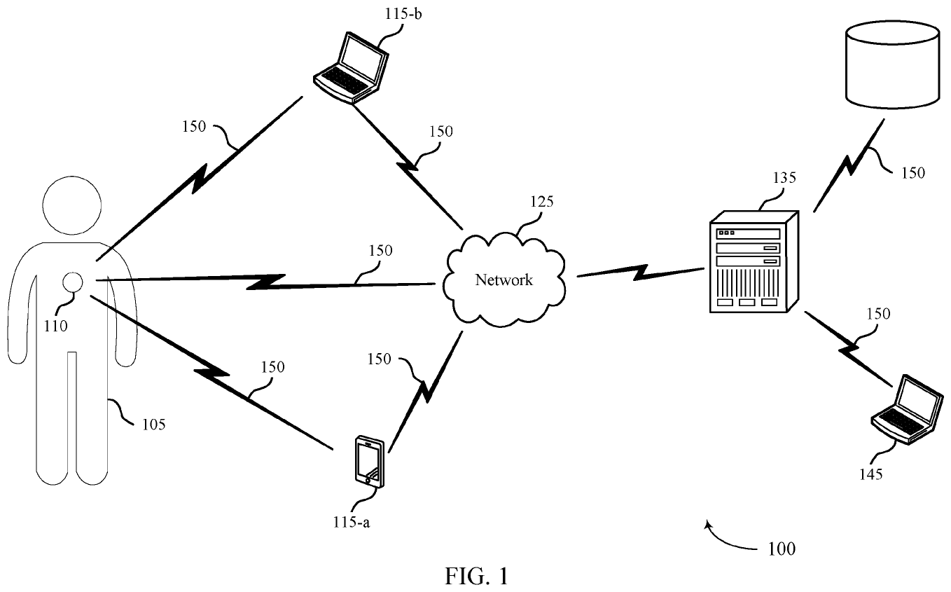

FIG. 1 illustrates an example of a wireless patient monitoring system 100 in accordance with aspects of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105. In some cases, the medical device 110 may include a sensor associated with operating parameters of a ventilator. In such cases, the medical device 110 may be an example of the ventilator. For example, the medical device 110 may include a gas concentration sensor, a flow sensor, a pressure sensor, or a volume sensor.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.). In some examples, the medical device 110 may be coupled to a ventilator. The data collected by the ventilator may be wirelessly transmitted to either the computing devices 115 or to the remote computing device 145 (via the network 125 and central station 135). Data transmission may occur via, for example, frequencies appropriate for a personal area network (such as Bluetooth, BLE, or IR communications) or local (e.g., WLAN) or WAN frequencies.

Computing device 115-a may be a wireless device such as a tablet, cellular phone, personal digital assistant (PDA), a dedicated receiver, or other similar device or a spatially distributed network of devices configured to receive signals from the medical device 110. Computing device 115-b may be a wireless laptop computer, a clinician Workstation on Wheels, or a smart hospital bed configured to receive signals from the medical device 110. The computing devices 115 may be in communication with a central station 135 via network 125.

The medical device 110 may also communicate directly with the central station 135 via the network 125. The central station 135 may be a server or a central nurse station located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In accordance with various embodiments, methods and apparatuses are described for a remote ventilation dashboard system. In some systems, the increased amount of data from one or more medical devices 110 associated with each patient 105 may make it difficult for clinicians to hear and differentiate patient alarms. In some cases, the settings of the ventilator may be changed multiple times throughout the day, and the clinicians may request to be notified when a setting is changed. In such cases, a remote ventilation dashboard may be utilized to notify clinicians of setting changes, patient alarms, or both, thereby improving the response time of the clinician and reducing an amount of time a patient may be at risk.

A ventilation dashboard server may display physiological data in a user interface (e.g., via computing devices 115 or remote computing device 145) by identifying a table from a set of tables. The table defines a ratio or a set of ratios between a first set of values for a first physiological parameter and a second set of values for a second physiological parameter. The ventilation dashboard server may display, in the user interface, a first row including the first set of values and a second row including the second set of values. In some cases, the first row and the second row are adjacent (e.g., arranged next to each other or near each other within the user interface) and aligned with each other (e.g., each row may be the same length and may be aligned such that the two rows start at the same location on the user interface and end at the same location on the user interface). The ventilation dashboard server may receive measured values associated with the first physiological parameter and the second physiological parameter from the medical device 110 (e.g., a ventilator).

Based on the received measured values, the ventilation dashboard server may identify a first display range for the first set of values and a second display range for the second set of values. The ventilation dashboard server may display a first visual indicator in the first row and second row that highlights the first display range and second display range. The ventilation dashboard server may also display a second visual indicator in the first row and second row that changes the color of the first visual indicator based on whether the first display range and the second display range overlap.

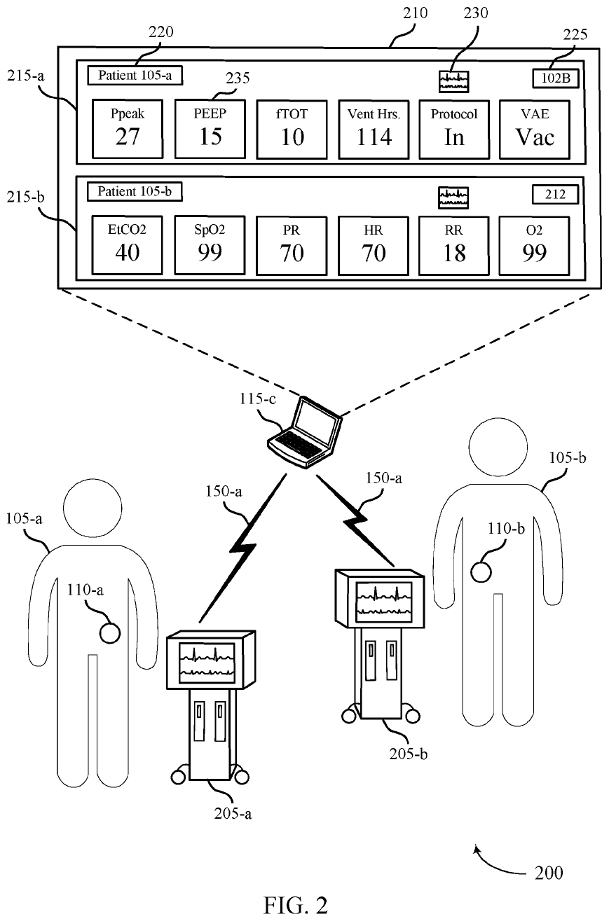

FIG. 2 illustrates an example of a system 200 for displaying physiological data in a user interface that supports a remote ventilation dashboard system in accordance with aspects of the present disclosure. In some examples, system 200 may implement aspects of wireless patient monitoring system 100. The system 200 may include a first patient 105-a wearing, carrying, or otherwise coupled with a medical device 110-a and a second patient 105-b wearing, carrying, or otherwise coupled with a medical device 110-b. The medical device 110-a may be an example of a medical sensor coupled with a ventilator 205-a. The medical device 110-b may be an example of a medical sensor coupled with a ventilator 205-b.

The ventilator 205-a and ventilator 205-b may transmit signals via wireless communications links 150-a to computing device 115-c. Computing device 115-c may be an example of computing device 115 or remote computing device 145 as described in reference to FIG. 1. The ventilator 205-a and ventilator 205-b may be directly coupled with the computing device 115-c. In some systems, ventilation settings and parameters may be documented manually, which may be time consuming and prone to transcription errors. System 200 may stream ventilation data from ventilator 205-a and ventilator 205-b to EMR systems, thereby reducing an amount of time on documentation and increasing an amount of time on patient care. In such cases, the system 200 may document the ventilation settings and parameters with increased accuracy, thereby increasing the efficiency and accuracy of patient care. In some cases, the ventilator 205-a and ventilator 205-b may transmit over 60 different settings and over 40 parameters via wireless communications links 150-a. The data transmitted by ventilator 205-a and ventilator 205-b may be an example of health level seven (HL7) data.

The computing device 115-c may display a patient care tile screen 210. The patient care tile screen 210 may include multiple patient tiles 215 for multiple patients 105. In some cases, the data displayed in the patient care tile screen 210 may be configurable for each patient 105. For example, the patient care tile screen 210 may include a first patient tile 215-a and a second patient tile 215-b. The first patient tile 215-a may correspond to information and medical data associated with the first patient 105-a, and the second patient tile 215-b may correspond to information and medical data associated with the second patient 105-b.

The first patient tile 215-a may include patient identification 220, patient locator 225, status indicator 230, and physiological parameter tiles 235. The patient identification 220 may include a name of the patient 105-a, and the patient locator 225 may include a location of the patient within a hospital, nursing home, or clinic (e.g., a bed number, a room number, a floor number, etc.). The status indicator 230 may indicate whether the medical device 110-a is actively receiving measured values associated with physiological parameters. For example, the status indicator 230 may be displayed above the physiological parameter tile 235 for a medical device 110-a that is transmitting values associated with the physiological parameter via wireless communications links 150-a. In some cases, the status indicator 230 may be an example of a ventilation dashboard icon that may automatically populate the patient tile 215-a after the ventilator 205-a is linked to the patient 105-a. The ventilation dashboard server may select physiological parameter tiles 235 and the patient care tile screen 210 may update to display a ventilation dashboard.

The physiological parameter tiles 235 may display a value associated with the physiological parameter. The physiological parameter tiles 235 may be an example of ventilator parameter tiles to display a measured value of peak pressure (Pₚₑₐₖ), a measured value of a positive end-expiratory pressure (PEEP), a value of a high respiratory rate alarm (f_{TOT}), or a combination thereof. In some cases, the physiological parameter tiles 235 may be an example of dashboard parameter tiles to display a time on the ventilator 205-a, a protocol, a ventilation associated event (VAE), or a combination thereof. In some examples, the physiological parameter tiles 235 may be an example of a tile to display a measured value of an end-tidal CO₂ (EtCO₂), a value of SpO₂, a value of a normal resting heart rate (PR), a measured value of the heart rate (HR), a measured value of a respiratory rate (RR), or a combination thereof.

The physiological parameter tile 235 indicating the VAE may indicate a VAE triggering event, a VAE triggering alarm, a VAE status, or a combination thereof. In some cases, the PEEP may be tracked over time. For example, if the PEEP exceeds a threshold, the physiological parameter tile 235 may indicate that a first day of a ventilation associated condition (VAC) may be met, and the physiological parameter tile 235 indicating the VAE may be updated to indicate VD1. If the PEEP exceeds the threshold for two consecutive days, the physiological parameter tiles 235 indicating the VAE may be updated to indicate VAC. The baseline stability of the PEEP may be set based on an average measured value of the PEEP over two days. The physiological parameter tile 235 indicating the protocol may notify the clinician whether the patient 105 is in protocol or out of protocol.

The patient care tile screen 210 may be an example of a home screen for the remote ventilation dashboard system. In some cases, an alert may be transmitted to the patient care tile screen 210 based on the measured physiological parameter exceeding a threshold. In such cases, a color of the corresponding physiological parameter tile 235 may be updated to display a red color if the physiological parameter exceeds the threshold. In other examples, the color of the physiological parameter tile 235 may be updated to display a green color if the physiological parameter is below the threshold. In some cases, the computing device 115-c may emit an audible alert based on the color of the physiological parameter tile 235 updating to display the red color.

A priority of the ventilator alarms (e.g., PEEP and f_{TOT}) may be greater than a priority of a dashboard notification (e.g., the time on the ventilator 205-a, the protocol, and the VAE). The patient tiles 215 may be an adjunct in patient assessment. For example, the patient care tile screen 210 may allow the clinician to make patient specific changes on the patient care tile screen 210 (e.g., dashboard). The clinician may update a list of alert thresholds via the patient care tile screen 210, locate a variable to change, or update a minimum and maximum value associated with the alert. In such cases, the computing device 115-c may customize alarm thresholds and transmit the customized alarms to devices of other clinicians.

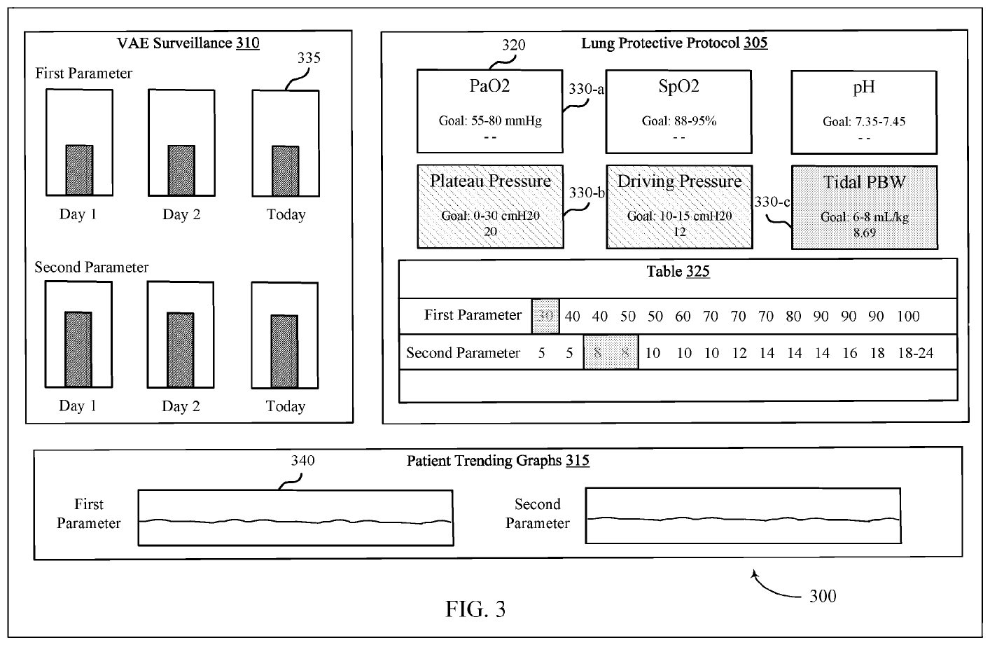

FIG. 3 illustrates an example of a ventilation dashboard 300 that supports remote ventilation dashboard system in accordance with aspects of the present disclosure. In some examples, the ventilation dashboard 300 may implement aspects of wireless patient monitoring system 100. The ventilation dashboard 300 may include a lung protective protocol dashboard 305, a VAE surveillance dashboard 310, and patient trending graphs 315. The ventilation dashboard 300 may illustrate information for a single patient.

The lung protective protocol dashboard 305 may include physiological parameter tiles 320, which may be examples of physiological parameter tiles 235 as described in reference to FIG. 2. Each physiological parameter tile 320 may indicate a measured value of the physiological parameter and an optimum value range (e.g., goal) of the physiological parameter. The physiological parameter tiles 320 may be an example of parameter tiles to display partial pressure of oxygen (PaO₂), SPO₂, pH, plateau pressure, driving pressure, tidal predicated body weight (PBW), or a combination thereof.

In some cases, the physiological parameter tiles 320 may display a first visual indicator 330-a (e.g., no color) if the physiological parameter tiles 320 are not actively receiving values associated with the physiological parameter. In some cases, the physiological parameter tiles 320 may display a second visual indicator 330-b (e.g., a green color) if the physiological parameter tiles 320 are actively receiving values associated with the physiological parameter and the received value is within the optimum value range. The physiological parameter tiles 320 may display a third visual indicator 330-c (e.g., a red color) if the physiological parameter tiles 320 are actively receiving values associated with the physiological parameter and the received value is below or above the optimum value range.

The physiological parameter tiles 320 may display the second visual indicator 330-b or the third visual indicator 330-c to communicate if the patient is inside or outside of a user defined protocol target. For example, the second visual indicator 330-b may indicate that the patient is inside the user defined protocol target, and the third visual indicator 330-c may indicate that the patient is outside the user defined protocol target. The lung protective protocol dashboard 305 may be based on an acute respiratory distress syndrome net (ARDSNet) protocol.

The lung protective protocol dashboard 305 may include a table 325. The table 325 may be an example of an ARDS surveillance table. The table 325 may display values associated with a first parameter and second parameter. In some examples, the first physiological parameter is an example of a FiO2, and the second physiological parameter is an example of PEEP. The table 325 may derived from the ARDSNet protocol where a user can select a first table (e.g., higher PEEP table) or a second table (e.g., lower PEEP table).

The table 325 may display a first visual indicator or a second visual indicator to assist with PEEP optimization. In some examples, a ventilation dashboard server may display, in a user interface (e.g., ventilation dashboard 300), a tile (e.g., physiological parameter tile 320) configured to indicate whether a first display range of the table 325 and a second display range of table 325 overlap. In some case, the physiological parameter tiles 320 may be updated based on a manual entry of data. In such cases, the visual indicators 330 for the physiological parameter tiles 320 may be updated automatically (e.g., automatically refreshed).

The lung protective protocol dashboard 305 may be updated frequently with data from the ventilator. For example, the lung protective protocol dashboard 305 may be updated every hour with new data coming directly from the ventilator. In other examples, the lung protective protocol dashboard 305 may be updated every 15 minutes. In such cases, the table 325 may be updated (e.g., refreshed) every hour, every 15 minutes, or other frequent amounts of time. The duration between updates may be based on a time that the lung protective protocol dashboard 305 is linked to the patient (e.g., the ventilator associated with the patient).

The VAE surveillance dashboard 310 may include bar graphs 335 for the first parameter and the second parameter. The bar graphs 335 may illustrate a minimum daily value of the first parameter for the past two days (e.g., Day 1 and Day 2) and a minimum daily value of the first parameter for the current day (e.g., Today). The bar graphs 335 may illustrate the minimum daily value of the second parameter for the past two days (e.g., Day 1 and Day 2) and the minimum daily value of the second parameter for the current day (e.g., Today). The VAE surveillance dashboard 310 may be based on the Center for Disease Control (CDC) guidelines. In some cases, the lung protective protocol dashboard 305 and the VAE surveillance dashboard 310 may be updated every 15 minutes based on measured values associated with the physiological parameters.

The bar graphs 335 may be determined based on determining a minimum value of the first parameter and the second parameter. In some cases, if the first parameter or second parameter exceeds a threshold, a first day of the VAC may be met and the physiological parameter tile indicating the VAE may be updated to indicate VD1. In other examples, if the first parameter or second parameter exceeds the threshold for two consecutive days, the physiological parameter tile indicating the VAE may be updated to indicate VAC. In such cases, the color of the bar graph 335 for the current day may change from a first color to a second color.

The VAE surveillance dashboard 310 may be updated frequently with data from the ventilator. For example, the VAE surveillance dashboard 310 may be updated every 15 minutes with new data coming directly from the ventilator. In such cases, the bar graph 335 for the current day may be updated (e.g., refreshed) every 15 minutes. The duration between updates may be based on a time that the VAE surveillance dashboard 310 is linked to the patient (e.g., the ventilator associated with the patient).

The patient trending graphs 315 may include trend graphs 340 for a first parameter and second parameter. The trend graphs 340 may each be an example of ventilator trend graphs for a first parameter and a second parameter. In some cases, the patient trending graphs 315 may include trend graphs for mean airway passage, peak inspiratory pressure (PIP), tidal volume, tidal PWB, or a combination thereof. In some cases, the ventilation dashboard server may generate a notification associated with the trend graph 340 exceeding a customizable threshold. The threshold may be displayed on the trend graphs 340.

The patient trending graphs 315 may be updated frequently with data from the ventilator. For example, the patient trending graphs 315 may be updated every hour with new data coming directly from the ventilator. In such cases, the trend graphs 340 may be updated (e.g., refreshed) every hour. The duration between updates may be based on a time that the patient trending graphs 315 are linked to the patient (e.g., the ventilator associated with the patient). In such cases, the refresh (e.g., update) times may be based on a time that the ventilation dashboard 300 is linked to the patient.

In some cases, the ventilation dashboard 300 may be configurable. For example, settings of the ventilation dashboard 300 may be set and parameterized. Settings and parameters of the ventilation dashboard 300 may include driving pressure, dynamic compliance, dynamic resistance, FiO₂, mean airway pressure, P/F ratio, PaCO₂, PaO₂, PIP, PEEP, pH, plateau pressure, S/F ratio, SpO₂, static lung compliance, tidal PBW, and tidal volume.

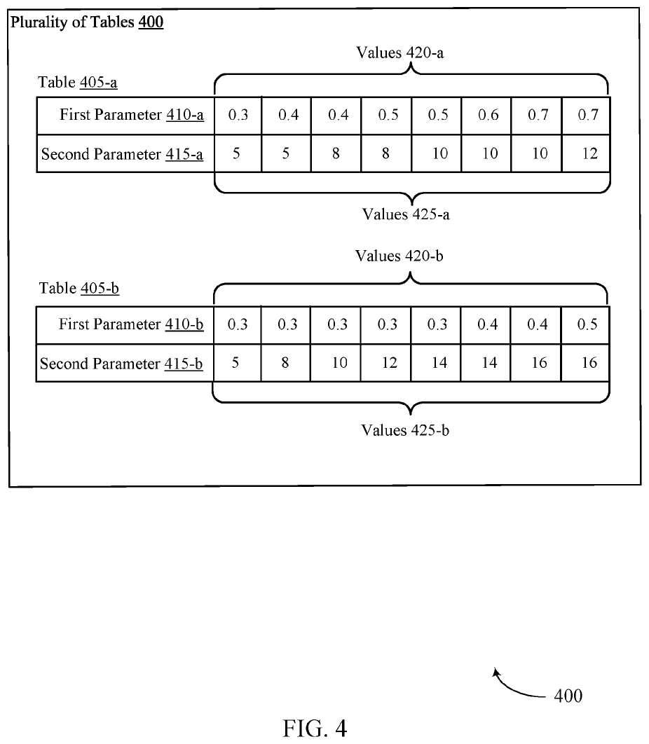

FIG. 4 illustrates an example of a plurality of tables 400 that supports remote ventilation dashboard system in accordance with aspects of the present disclosure. The plurality of tables 400 may include a first table 405-a and a second table 405-b. The first table 405-a may include a first row indicating values 420-a of a first parameter 410-a and a second row indicating values 425-a of a second parameter 415-a. The second table 405-b may include a first row indicating values 420-b of a first parameter 410-b and a second row indicating values 425-b of a second parameter 415-b.

The first table 405-a and the second table 405-b may be examples of a medically, agreed upon, source tables (e.g., reference tables from literature) to choose from. For example, the ventilation dashboard server may receive a user selection of either the first table 405-a or the second table 405-b. In some cases, the ventilation dashboard server may receive a user selection of the first table 405-a. In such cases, the first table 405-a may be identified from the plurality of tables 400. The first table 405-a may define a ratio (or a set of ratios) between the values 420-a (e.g., first set of values) for the first parameter 410-a (e.g., first physiological parameter) and the values 425-a (e.g., second set of values) for the second parameter 415-a (e.g., second physiological parameter). As explained above, the ratios of such values may provide insight regarding the presence of a physiological event as compared to the individual values measured and assessed separately. For example, a measured value of 0.3 for the first parameter 410-a, taken along, may not be indicative to a clinician of a physiological event. However, a measured value of 0.3 for the first parameter 410-a taken together with a measured value of 5 for the second parameter 415-a may indicate that the ratio of these two parameters is within the predefined ratio defined by table 420-a (which may indicate that the patient is within normal range). In a similar fashion, a same measured value of 0.3 for the first parameter 410-a, taken together with a measured value of 10 for the second parameter 415-a, may indicate that the ratio of these two parameters is outside of the predefined ratio threshold (which may indicate that the patient is suffering from an adverse physiological event). Therefore, when assessing physiological parameters where a comparison between the parameters is just as critical (or more critical) than the individual measurements of the parameters, a more nuanced visual interface or method of displaying the measured parameters may be beneficial. For example, triggering an alarm (or visual indicator) when one of the first parameter 410-a or the second parameter 415-a falls above or below a threshold may not provide the clinician with relevant or actionable data. Instead, as described in detail herein with respect to several examples, displaying indicators of whether a pair of measured values falls within a predefined ratio (based on a ratios defined in a reference table) may be an improvement to a user interface that facilitates quick and accurate assessment of combinations of measured physiological values.

In some examples, the first parameters 410-a and 410-b may include the FiO2, and the second parameters 415-a and 415-b may include the PEEP. The first table 405-a may be an example of a table indicating low PEEP values (e.g., values 425-a) and high FiO2 values (e.g., values 420-a). The second table 405-b may be an example of a table indicating high PEEP values (e.g., values 425-b) and low FiO2 values (e.g., values 420-b). In some examples, the ventilation dashboard server may receive a user selection of the first table 405-a if the user selects to display the table indicating low PEEP values (e.g., values 425-a) and high FiO2 values (e.g., values 420-a). In other examples, the ventilation dashboard server may receive a user selection of the second table 405-b if the user selects to display the table indicating high PEEP values (e.g., values 425-b) and low FiO2 values (e.g., values 420-b).

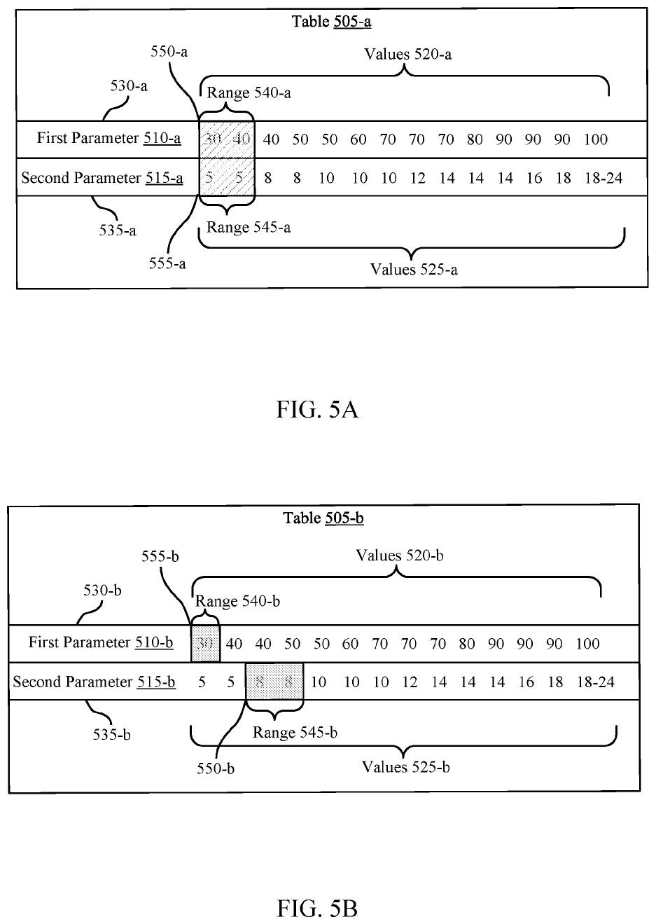

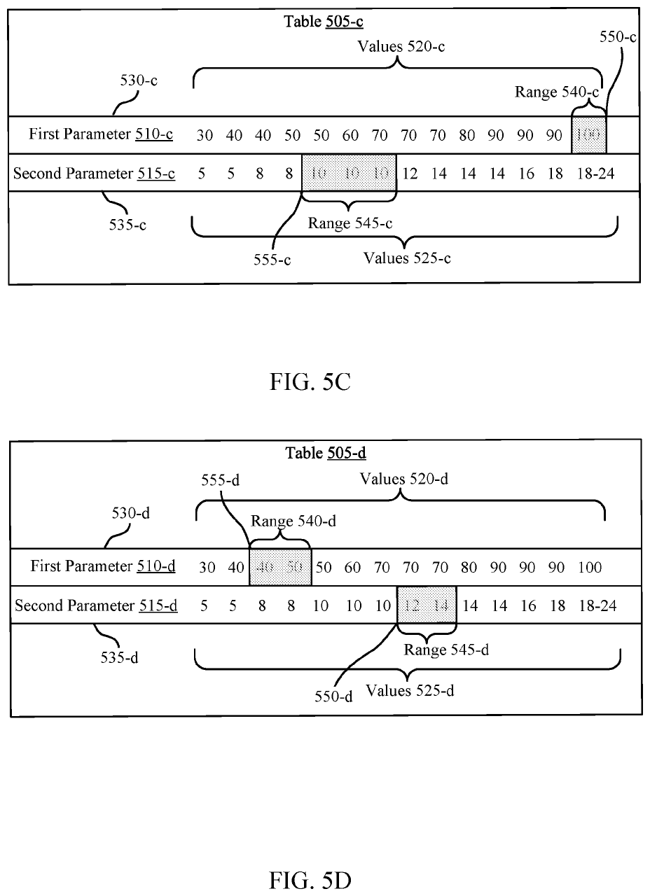

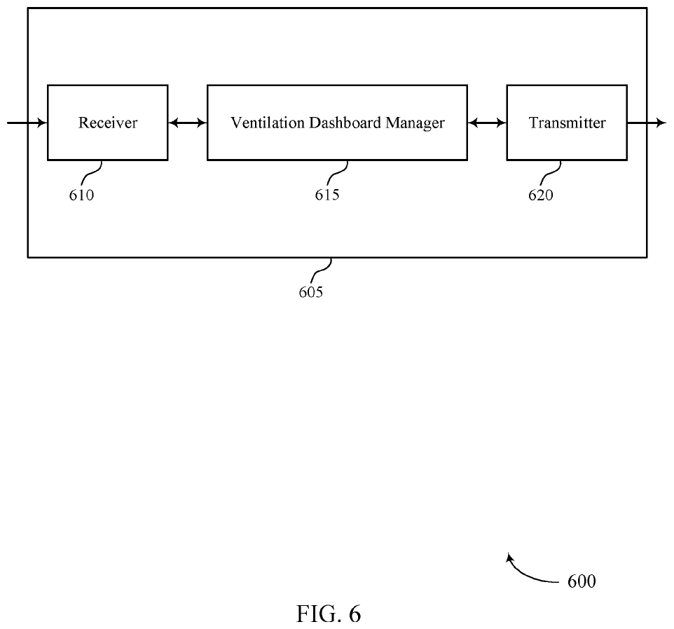

FIG. 5A illustrates an example of a table 505-a that supports remote ventilation dashboard system in accordance with aspects of the present disclosure. Table 505-a may be an example of table 325 as described in reference to FIG. 3. First parameter 510-a and second parameter 515-a may be an example of first parameter 410 and second parameter 415 as described in reference to FIG. 4.

Based on the selection of a first table or second table from the plurality of tables, the ventilation dashboard server may display, within the table 505-a, a first row 530-a including values 520-a (e.g., first set of values) for the first parameter 510-a (e.g., first physiological parameter). The ventilation dashboard server may display, within the table 505-a, a second row 535-a including values 525-a (e.g., second set of values) for the second parameter 515-a (e.g., second physiological parameter). The values 520-a may correspond to the values of the first parameter in the selected first table, and the values 525-a may correspond to the values of the second parameter in the selected first table. The first row 530-a and the second row 535-a may be adjacent and aligned with each other such that each value of the values 520-a may be aligned with each value of the values 525-a.

The ventilation dashboard server may receive, from a medical device associated with the patient, a first measured value associated with the first parameter 510-a and a second measured value associated with the second parameter 515-a. In some examples, the measured values may be received directly from a ventilator. For example, the ventilation dashboard server may receive a value of 35 for the first parameter 510-a and a value of 5 for the second parameter 515-a.

In the first row 530-a, a first display range 540-a of the values 520-a for the first parameter 510-a may be identified. The first display range 540-a may be based on the received first measured value (e.g., 35). In some examples, the ventilation dashboard server may determine that the first measured value is a value between a first value and a second value of the values 520-a in the first row 530-a. In such cases, the ventilation dashboard server may highlight the first value (e.g., 30) and the second value (e.g., 40) of the values 520-a in the first row 530-a based on determining that the first measured value (e.g., 35) is a value between the first value and the second value. The first display range 540-a may include the first value of 30 and the second value 40 of the values 520-a.

In some cases, a second display range 545-a of the values 525-a for the second parameter 515-a may be identified in the second row 535-a. The second display range 545-a may be based on the received second measured value (e.g., 5). In some examples, the ventilation dashboard server may determine that the second measured value is a value of the values 525-a. For example, the second measured value matches at least a value of the values 525-a. In such cases, the ventilation dashboard server may highlight the value (e.g., 5) of the values 525-a in the second row 535-a based on determining that the second measured value is a value of the values 525-a. For example, the ventilation dashboard server may highlight a subset of the values 525-a in the second row 535-a that matches the second measured value. In some cases, the subset of the values 525-a may be duplicated values. For example, the second display range 545-a may include each value of 5 of the values 525-a.

The ventilation dashboard server may display, in the first row 530-a and the second row 535-a, a visual indicator 550-a (e.g., first visual indicator) that highlights or otherwise indicates on the user interface the first display range 540-a and the second display range 545-a. In some examples, the ventilation dashboard server may display, in the first row 530-a and the second row 535-a, a visual indicator 555-a (e.g., second visual indicator such as changing a color of the highlighted ranges), that updates a characteristic of the visual indicator 550-a based on whether the first display range 540-a and the second display range 545-a visually overlap between the first row 530-a and second row 535-a.

For example, displaying the visual indicator 555-a may include highlighting the first display range 540-a and the second display range 545-a a first color (e.g., green) based on the first display range 540-a and the second display range 545-a overlapping. In such cases, the corresponding tile on the lung protective protocol may be updated to display the first color. Displaying the visual indicator 550-a and the visual indicator 555-a may enable the ventilation dashboard to provide a quick and efficient way to identify data that is relevant or beneficial to the clinician at the time of reviewing. In some examples, determining whether the first display range 540-a and the second display range 545-a overlap and displaying a visual indicator 555-a based on the overlap may provide an efficient way to identify patients that may not be experiencing a significant physiological event if the tile on the lung protective protocol displays the first color.

FIG. 5B illustrates an example of a table 505-b that supports remote ventilation dashboard system in accordance with aspects of the present disclosure. Table 505-b may be an example of table 325 and table 505-a as described in reference to FIGS. 3 and 5A. First parameter 510-b and second parameter 515-b may be an example of first parameter and second parameter as described in reference to FIGS. 4 and 5.

The ventilation dashboard server may receive, from a medical device associated with the patient, a first measured value associated with the first parameter 510-b and a second measured value associated with the second parameter 515-b. In some examples, the measured values may be received directly from a ventilator. For example, the ventilation dashboard server may receive a value of 30 for the first parameter 510-b and a value of 8 for the second parameter 515-a.

In the first row 530-b, a first display range 540-b of the values 520-b for the first parameter 510-b may be identified. The first display range 540-b may be based on the received first measured value (e.g., 30). In some examples, the ventilation dashboard server may determine that the first measured value is a value of the values 520-b. For example, the first measured value matches at least a value of the values 520-b. In such cases, the ventilation dashboard server may highlight the value (e.g., 30) of the values 520-b in the first row 530-b based on determining that the first measured value is a value of the values 520-b. For example, the ventilation dashboard server may highlight a subset of the values 520-b in the first row 530-b that matches the first measured value. In some cases, the subset of the values 520-b may be a single value. For example, the first display range 540-b may include the value of 30 of the values 520-b.

In some cases, a second display range 545-b of the values 525-b for the second parameter 515-b may be identified in the second row 535-b. The second display range 545-b may be based on the received second measured value (e.g., 8). In some examples, the ventilation dashboard server may determine that the second measured value is a value of the values 525-b. For example, the second measured value matches at least a value of the values 525-b. In such cases, the ventilation dashboard server may highlight the value (e.g., 8) of the values 525-b in the second row 535-b based on determining that the second measured value is a value of the values 525-b. For example, the ventilation dashboard server may highlight a subset of the values 525-b in the second row 535-b that matches the second measured value. In some cases, the subset of the values 525-b may be duplicated values. For example, the second display range 545-b may include each value of 8 of the values 525-b.

The ventilation dashboard server may display, in the first row 530-b and the second row 535-b, a visual indicator 550-b (e.g., first visual indicator) that highlights the first display range 540-b and the second display range 545-b. In some examples, the ventilation dashboard server may display, in the first row 530-b and the second row 535-b, a visual indicator 555-b (e.g., second visual indicator), that updates a characteristic of the visual indicator 550-b based on whether the first display range 540-b and the second display range 545-b visually overlap between the first row 530-b and second row 535-b.

For example, displaying the visual indicator 555-b may include highlighting the first display range 540-b and the second display range 545-b a second color (e.g., red) based on the first display range 540-b and the second display range 545-b being non-overlapping. In such cases, the corresponding tile on the lung protective protocol may be updated to display the second color. Displaying the visual indicator 550-b and the visual indicator 555-b may enable the ventilation dashboard to provide a quick and efficient way to identify data that is relevant or beneficial to the clinician at the time of reviewing. In some examples, determining whether the first display range 540-b and the second display range 545-b overlap and displaying the visual indicator 555-b based on the overlap may provide an efficient way to identify patients that may be experiencing a significant physiological event if the tile on the lung protective protocol displays the second color, thereby reducing the amount of time a patient may be at risk.

FIG. 5C illustrates an example of a table 505-c that supports remote ventilation dashboard system in accordance with aspects of the present disclosure. Table 505-c may be an example of table 325 and table 505-b as described in reference to FIGS. 3 and 5B. First parameter 510-c and second parameter 515-c may be an example of first parameter and second parameter as described in reference to FIGS. 4 and 5.

The ventilation dashboard server may receive, from a medical device associated with the patient, a first measured value associated with the first parameter 510-c and a second measured value associated with the second parameter 515-c. In some examples, the measured values may be received directly from a ventilator. For example, the ventilation dashboard server may receive a value of 100 for the first parameter 510-c and a value of 10 for the second parameter 515-c.

In the first row 530-c, a first display range 540-c of the values 520-c for the first parameter 510-c may be identified. The first display range 540-c may be based on the received first measured value (e.g., 100). In some examples, the ventilation dashboard server may determine that the first measured value is a value of the values 520-c. For example, the first measured value matches at least a value of the values 520-c. In such cases, the ventilation dashboard server may highlight the value (e.g., 100) of the values 520-c in the first row 530-c based on determining that the first measured value is a value of the values 520-c. For example, the ventilation dashboard server may highlight a subset of the values 520-c in the first row 530-c that matches the first measured value. In some cases, the subset of the values 520-c may be a single value. For example, the first display range 540-c may include the value of 100 of the values 520-c.

In some cases, a second display range 545-c of the values 525-c for the second parameter 515-c may be identified in the second row 535-c. The second display range 545-c may be based on the received second measured value (e.g., 10). In some examples, the ventilation dashboard server may determine that the second measured value is a value of the values 525-c. For example, the second measured value matches at least a value of the values 525-c. In such cases, the ventilation dashboard server may highlight the values (e.g., 10) of the values 525-c in the second row 535-c based on determining that the second measured value is a value of the values 525-c. For example, the ventilation dashboard server may highlight a subset of the values 525-c in the second row 535-c that matches the second measured value. In some cases, the subset of the values 525-c may be duplicated values. For example, the second display range 545-c may include each value of 10 of the values 525-c.

The ventilation dashboard server may display, in the first row 530-c and the second row 535-c, a visual indicator 550-c (e.g., first visual indicator) that highlights the first display range 540-c and the second display range 545-c. In some examples, the ventilation dashboard server may display, in the first row 530-c and the second row 535-c, a visual indicator 555-c (e.g., second visual indicator), that updates a characteristic of the visual indicator 550-c based on whether the first display range 540-c and the second display range 545-c visually overlap between the first row 530-c and second row 535-c.

For example, displaying the visual indicator 555-c may include highlighting the first display range 540-c and the second display range 545-c the second color (e.g., red) based on the first display range 540-c and the second display range 545-c being non-overlapping. In such cases, the corresponding tile on the lung protective protocol may be updated to display the second color. Displaying the visual indicator 550-c and the visual indicator 555-c may enable the ventilation dashboard to provide a quick and efficient way to identify data that is relevant or beneficial to the clinician at the time of reviewing. In some examples, determining whether the first display range 540-c and the second display range 545-c overlap and displaying the visual indicator 555-c based on the overlap may provide an efficient way to identify patients that may be experiencing a significant physiological event if the tile on the lung protective protocol displays the second color, thereby reducing the amount of time a patient may be at risk.

FIG. 5D illustrates an example of a table 505-d that supports remote ventilation dashboard system in accordance with aspects of the present disclosure. Table 505-d may be an example of table 325 and table 505-a as described in reference to FIGS. 3 and 5A. First parameter 510-d and second parameter 515-d may be an example of first parameter and second parameter as described in reference to FIGS. 4 and 5.

The ventilation dashboard server may receive, from a medical device associated with the patient, a first measured value associated with the first parameter 510-d and a second measured value associated with the second parameter 515-d. In some examples, the measured values may be received directly from a ventilator. For example, the ventilation dashboard server may receive a value of 44 for the first parameter 510-d and a value of 13 for the second parameter 515-d.

In the first row 530-d, a first display range 540-d of the values 520-a for the first parameter 510-d may be identified. The first display range 540-d may be based on the received first measured value (e.g., 44). In some examples, the ventilation dashboard server may determine that the first measured value is a value between a first value and a second value of the values 520-d in the first row 530-d. In such cases, the ventilation dashboard server may highlight the first value (e.g., 40) and the second value (e.g., 50) of the values 520-d in the first row 530-d based on determining that the first measured value (e.g., 44) is a value between the first value and the second value. The first display range 540-d may include the first value of 40 and the second value 50 of the values 520-d.

In some cases, a second display range 545-d of the values 525-d for the second parameter 515-d may be identified in the second row 535-d. The second display range 545-d may be based on the received second measured value (e.g., 13). In some examples, the ventilation dashboard server may determine that the second measured value is a value between a first value and a second value of the values 525-d in the second row 535-d. In such cases, the ventilation dashboard server may highlight the first value (e.g., 12) and the second value (e.g., 14) of the values 525-d in the second row 535-d based on determining that the second measured value (e.g., 13) is a value between the first value and the second value. The second display range 545-d may include the first value of 12 and the second value 14 of the values 525-d.

The ventilation dashboard server may display, in the first row 530-d and the second row 535-d, a visual indicator 550-d (e.g., first visual indicator) that highlights the first display range 540-d and the second display range 545-d. In some examples, the ventilation dashboard server may display, in the first row 530-d and the second row 535-d, a visual indicator 555-d (e.g., second visual indicator), that updates a characteristic of the visual indicator 550-d based on whether the first display range 540-d and the second display range 545-d visually overlap between the first row 530-d and second row 535-d.

For example, displaying the visual indicator 555-d may include highlighting the first display range 540-d and the second display range 545-d the second color (e.g., red) based on the first display range 540-d and the second display range 545-d being non-overlapping. In such cases, the corresponding tile on the lung protective protocol may be updated to display the second color. Displaying the visual indicator 550-d and the visual indicator 555-d may enable the ventilation dashboard to provide a quick and efficient way to identify data that is relevant or beneficial to the clinician at the time of reviewing. In some examples, determining whether the first display range 540-d and the second display range 545-d overlap and displaying a visual indicator 555-d based on the overlap may provide an efficient way to identify patients that may be experiencing a significant physiological event if the tile on the lung protective protocol displays the second color, thereby reducing the amount of time a patient may be at risk.

FIG. 6 shows a block diagram 600 of a ventilation dashboard server 605 that supports remote ventilation dashboard system in accordance with aspects of the present disclosure. The ventilation dashboard server 605 may be an example of aspects of a server as described herein. The ventilation dashboard server 605 may include a receiver 610, a ventilation dashboard manager 615, and a transmitter 620. The ventilation dashboard server 605 may also include a processor. Each of these components may be in communication with one another (e.g., via one or more buses).

The receiver 610 may receive information such as packets, user data, or control information associated with various information channels (e.g., control channels, data channels, and information related to digital patient engagement, etc.). Information may be passed on to other components of the ventilation dashboard server 605. The receiver 610 may be an example of aspects of the transceiver 920 described with reference to FIG. 9. The receiver 610 may utilize a single antenna or a set of antennas.

The ventilation dashboard manager 615 may identify a first table from a set of tables, where the first table defines a ratio between a first set of values for a first physiological parameter and a second set of values for a second physiological parameter, display a first row including the first set of values for the first physiological parameter and a second row including the second set of values for the second physiological parameter, where the first row and the second row are adjacent and aligned with each other, receive, from a medical device associated with a patient, a first measured value associated with the first physiological parameter and a second measured value associated with the second physiological parameter, identify a first display range of the first set of values for the first physiological parameter based on the received first measured value, identify a second display range of the second set of values for the second physiological parameter based on the received second measured value, display, in the first row and in the second row, a first visual indicator that highlights the first display range in the first row and the second display range in the second row, and display, in the first row and in the second row, a second visual indicator that updates a characteristic of the first visual indicator for the first display range and the second display range based on whether the first display range and the second display range visually overlap between the first row and the second row. The ventilation dashboard manager 615 may be an example of aspects of the ventilation dashboard manager 910 described herein.

The ventilation dashboard manager 615, or its sub-components, may be implemented in hardware, code (e.g., software or firmware) executed by a processor, or any combination thereof. If implemented in code executed by a processor, the functions of the ventilation dashboard manager 615, or its sub-components may be executed by a general-purpose processor, a DSP, an application-specific integrated circuit (ASIC), a FPGA or other programmable logic device, discrete gate or transistor logic, discrete hardware components, or any combination thereof designed to perform the functions described in the present disclosure.

The ventilation dashboard manager 615, or its sub-components, may be physically located at various positions, including being distributed such that portions of functions are implemented at different physical locations by one or more physical components. In some examples, the ventilation dashboard manager 615, or its sub-components, may be a separate and distinct component in accordance with various aspects of the present disclosure. In some examples, the ventilation dashboard manager 615, or its sub-components, may be combined with one or more other hardware components, including but not limited to an input/output (I/O) component, a transceiver, a network server, another computing device, one or more other components described in the present disclosure, or a combination thereof in accordance with various aspects of the present disclosure.

The transmitter 620 may transmit signals generated by other components of the ventilation dashboard server 605. In some examples, the transmitter 620 may be collocated with a receiver 610 in a transceiver module. For example, the transmitter 620 may be an example of aspects of the transceiver 920 described with reference to FIG. 9. The transmitter 620 may utilize a single antenna or a set of antennas.

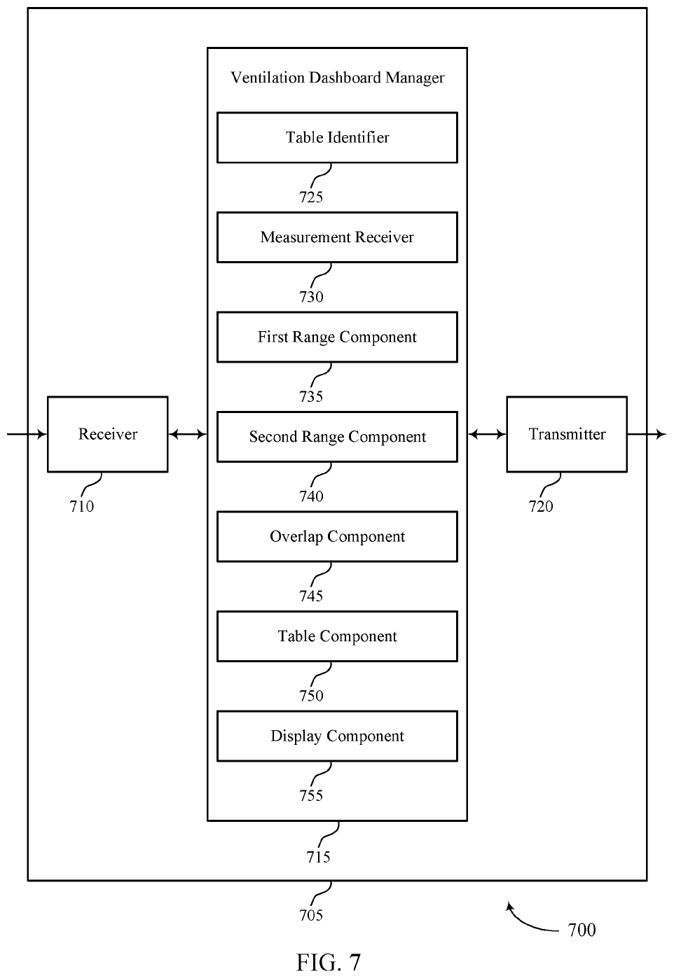

FIG. 7 shows a block diagram 700 of a ventilation dashboard server 705 that supports remote ventilation dashboard system in accordance with aspects of the present disclosure. The ventilation dashboard server 705 may be an example of aspects of a ventilation dashboard server 605, or a computing device 115 as described herein. The ventilation dashboard server 705 may include a receiver 710, a ventilation dashboard manager 715, and a transmitter 720. The ventilation dashboard server 705 may also include a processor. Each of these components may be in communication with one another (e.g., via one or more buses).

The receiver 710 may receive information such as packets, user data, or control information associated with various information channels (e.g., control channels, data channels, and information related to digital patient engagement, etc.). Information may be passed on to other components of the ventilation dashboard server 705. The receiver 710 may be an example of aspects of the transceiver 920 described with reference to FIG. 9. The receiver 710 may utilize a single antenna or a set of antennas.

The ventilation dashboard manager 715 may be an example of aspects of the ventilation dashboard manager 615 as described herein. The ventilation dashboard manager 715 may include a table identifier 725, a measurement receiver 730, a first range component 735, a second range component 740, an overlap component 745, a table component 750, and a display component 755. The ventilation dashboard manager 715 may be an example of aspects of the ventilation dashboard manager 910 described herein.

The table identifier 725 may identify a first table from a set of tables, where the first table defines a ratio between a first set of values for a first physiological parameter and a second set of values for a second physiological parameter.

The measurement receiver 730 may receive, from a medical device associated with a patient, a first measured value associated with the first physiological parameter and a second measured value associated with the second physiological parameter.

The first range component 735 may identify a first display range of the first set of values for the first physiological parameter based on the received first measured value.

The second range component 740 may identify a second display range of the second set of values for the second physiological parameter based on the received second measured value.

The overlap component 745 may display, in the first row and in the second row, a second visual indicator that updates a characteristic of the first visual indicator for the first display range and the second display range based on whether the first display range and the second display range visually overlap between the first row and the second row.

The table component 750 may display a first row including the first set of values for the first physiological parameter and a second row including the second set of values for the second physiological parameter, where the first row and the second row are adjacent and aligned with each other.

The display component 755 may display, in the first row and in the second row, a first visual indicator that highlights the first display range in the first row and the second display range in the second row.

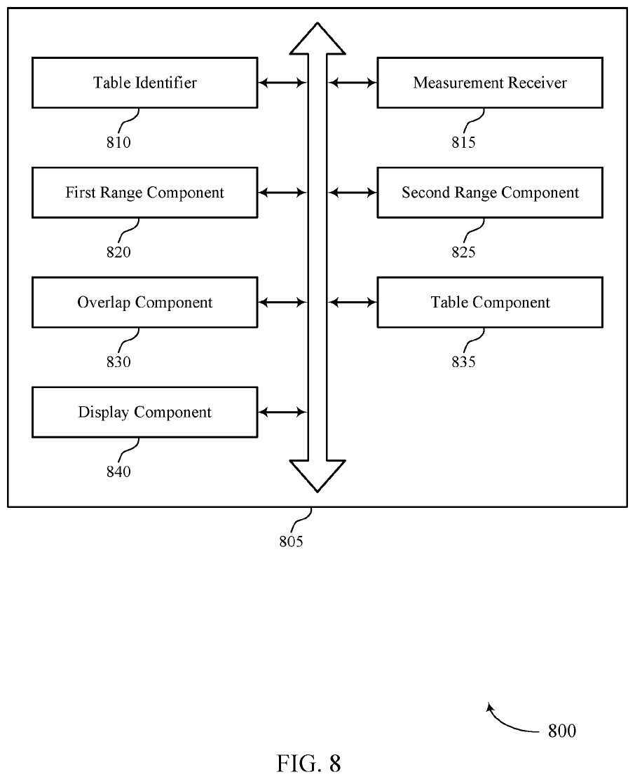

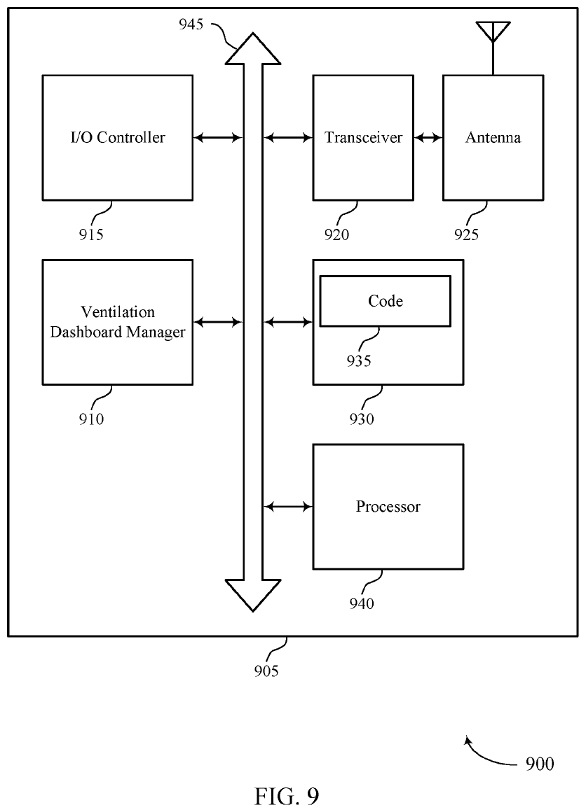

FIG. 8 shows a block diagram 800 of a ventilation dashboard manager 805 that supports remote ventilation dashboard system in accordance with aspects of the present disclosure. The ventilation dashboard manager 805 may be an example of aspects of ventilation dashboard manager 615, a ventilation dashboard manager 715, or a ventilation dashboard manager s described herein. The ventilation dashboard manager 805 may include a table identifier 810, a measurement receiver 815, a first range component 820, a second range component 825, an overlap component 830, a table component 835, and a display component 840. Each of these modules may communicate, directly or indirectly, with one another (e.g., via one or more buses).

The table identifier 810 may identify a first table from a set of tables, where the first table defines a ratio between a first set of values for a first physiological parameter and a second set of values for a second physiological parameter. In some examples, the table identifier 810 may receive a user selection of the first table from the set of tables.

The measurement receiver 815 may receive, from a medical device associated with a patient, a first measured value associated with the first physiological parameter and a second measured value associated with the second physiological parameter.

The first range component 820 may identify a first display range of the first set of values for the first physiological parameter based on the received first measured value. In some examples, the first range component 820 may highlight a subset of the first set of values in the first row that matches the first measured value. In some examples, the first range component 820 may determine that the first measured value is a value between a first value and a second value of the first set of values in the first row.

In some examples, the first range component 820 may highlight the first value and the second value of the first set of values in the first row based on determining that the first measured value is a value between the first value and the second value of the first set of values. In some cases, the subset of the first set of values or the subset of the second set of values includes duplicated values.

The second range component 825 may identify a second display range of the second set of values for the second physiological parameter based on the received second measured value. In some examples, the second range component 825 may highlight a subset of the second set of values in the second row that matches the second measured value.

In some examples, the second range component 825 may determine that the second measured value is a value between a first value and a second value of the second set of values in the second row. In some examples, the second range component 825 may highlight the first value and the second value of the second set of values in the second row based on determining that the second measured value is a value between the first value and the second value of the second set of values.

The overlap component 830 may display, in the first row and in the second row, a second visual indicator that updates a characteristic of the first visual indicator for the first display range and the second display range based on whether the first display range and the second display range visually overlap between the first row and the second row. In some examples, the overlap component 830 may highlight the first display range and the second display range a first color based on the first display range and the second display range overlapping. In some examples, the overlap component 830 may display, in the user interface, a tile configured to indicate whether the first display range and the second display range overlap based on the determination.

The table component 835 may display a first row including the first set of values for the first physiological parameter and a second row including the second set of values for the second physiological parameter, where the first row and the second row are adjacent and aligned with each other. In some cases, the first physiological parameter includes a fraction of inspired oxygen (FiO2) and the second physiological parameter includes a positive end-expiratory pressure (PEEP).

The display component 840 may display, in the first row and in the second row, a first visual indicator that highlights the first display range in the first row and the second display range in the second row. In some examples, the display component 840 may highlight the first display range and the second display range a second color based on the first display range and the second display range being non-overlapping.

FIG. 9 shows a diagram of a system 900 including a ventilation dashboard server 905 that supports remote ventilation dashboard system in accordance with aspects of the present disclosure. The ventilation dashboard server 905 may be an example of or include the components of ventilation dashboard server 605, ventilation dashboard server 705, or a ventilation dashboard server as described herein. The ventilation dashboard server 905 may include components for bi-directional voice and data communications including components for transmitting and receiving communications, including a ventilation dashboard manager 910, an I/O controller 915, a transceiver 920, an antenna 925, memory 930, and a processor 940. These components may be in electronic communication via one or more buses (e.g., bus 945).

The ventilation dashboard manager 910 may identify a first table from a set of tables, where the first table defines a ratio between a first set of values for a first physiological parameter and a second set of values for a second physiological parameter, display a first row including the first set of values for the first physiological parameter and a second row including the second set of values for the second physiological parameter, where the first row and the second row are adjacent and aligned with each other, receive, from a medical device associated with a patient, a first measured value associated with the first physiological parameter and a second measured value associated with the second physiological parameter, identify a first display range of the first set of values for the first physiological parameter based on the received first measured value, identify a second display range of the second set of values for the second physiological parameter based on the received second measured value, display, in the first row and in the second row, a first visual indicator that highlights the first display range in the first row and the second display range in the second row, and display, in the first row and in the second row, a second visual indicator that updates a characteristic of the first visual indicator for the first display range and the second display range based on whether the first display range and the second display range visually overlap between the first row and the second row.

The I/O controller 915 may manage input and output signals for the ventilation dashboard server 905. The I/O controller 915 may also manage peripherals not integrated into the ventilation dashboard server 905. In some cases, the I/O controller 915 may represent a physical connection or port to an external peripheral. In some cases, the I/O controller 915 may utilize an operating system such as iOS®, ANDROID®, MS-DOS®, MS-WINDOWS®, OS/2®, UNIX®, LINUX®, or another known operating system. In other cases, the I/O controller 915 may represent or interact with a modem, a keyboard, a mouse, a touchscreen, or a similar device. In some cases, the I/O controller 915 may be implemented as part of a processor. In some cases, a user may interact with the ventilation dashboard server 905 via the I/O controller 915 or via hardware components controlled by the I/O controller 915.

The transceiver 920 may communicate bi-directionally, via one or more antennas, wired, or wireless links as described above. For example, the transceiver 920 may represent a wireless transceiver and may communicate bi-directionally with another wireless transceiver. The transceiver 920 may also include a modem to modulate the packets and provide the modulated packets to the antennas for transmission, and to demodulate packets received from the antennas.

In some cases, the wireless device may include a single antenna 925. However, in some cases the device may have more than one antenna 925, which may be capable of concurrently transmitting or receiving multiple wireless transmissions.

The memory 930 may include RAM and ROM. The memory 930 may store computer-readable, computer-executable code 935 including instructions that, when executed, cause the processor to perform various functions described herein. In some cases, the memory 930 may contain, among other things, a BIOS which may control basic hardware or software operation such as the interaction with peripheral components or devices.

The processor 940 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 940 may be configured to operate a memory array using a memory controller. In other cases, a memory controller may be integrated into the processor 940. The processor 940 may be configured to execute computer-readable instructions stored in a memory (e.g., the memory 930) to cause the ventilation dashboard server 905 to perform various functions (e.g., functions or tasks supporting digital patient engagement).

The code 935 may include instructions to implement aspects of the present disclosure, including instructions to support managing digital communications with a patient. The code 935 may be stored in a non-transitory computer-readable medium such as system memory or other type of memory. In some cases, the code 935 may not be directly executable by the processor 940 but may cause a computer (e.g., when compiled and executed) to perform functions described herein.

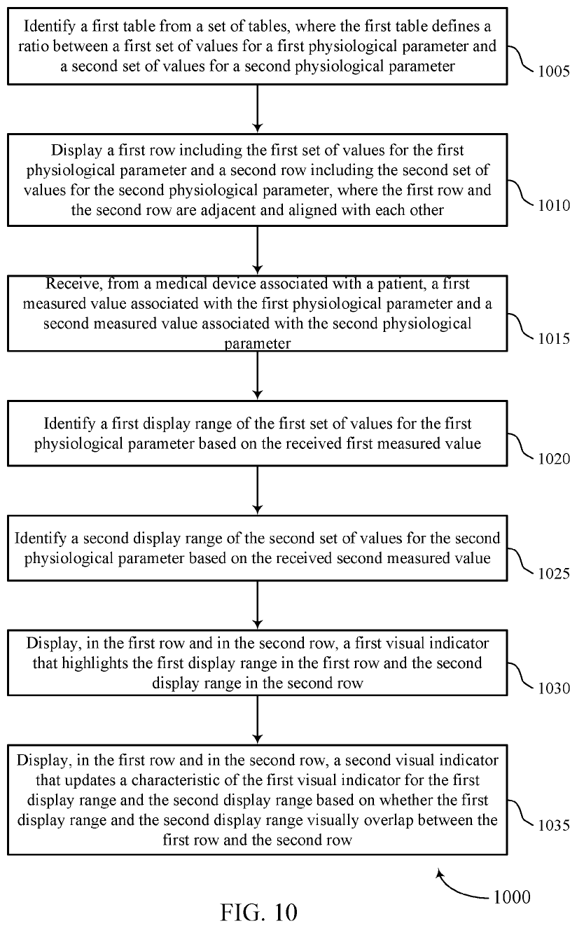

FIG. 10 shows a flowchart illustrating a method 1000 that supports remote ventilation dashboard system in accordance with aspects of the present disclosure. The operations of method 1000 may be implemented by a ventilation dashboard server or its components as described herein. For example, the operations of method 1000 may be performed by a default as described with reference to FIGS. 6 through 9. In some examples, a ventilation dashboard server may execute a set of instructions to control the functional elements of the ventilation dashboard server to perform the described functions. Additionally or alternatively, a ventilation dashboard server may perform aspects of the described functions using special-purpose hardware.

At 1005, the ventilation dashboard server may identify a first table from a set of tables, where the first table defines a ratio between a first set of values for a first physiological parameter and a second set of values for a second physiological parameter. The operations of 1005 may be performed according to the methods described herein. In some examples, aspects of the operations of 1005 may be performed by a table identifier as described with reference to FIGS. 6 through 9.

At 1010, the ventilation dashboard server may display a first row including the first set of values for the first physiological parameter and a second row including the second set of values for the second physiological parameter, where the first row and the second row are adjacent and aligned with each other. The operations of 1010 may be performed according to the methods described herein. In some examples, aspects of the operations of 1010 may be performed by a table component as described with reference to FIGS. 6 through 9.

At 1015, the ventilation dashboard server may receive, from a medical device associated with a patient, a first measured value associated with the first physiological parameter and a second measured value associated with the second physiological parameter. The operations of 1015 may be performed according to the methods described herein. In some examples, aspects of the operations of 1015 may be performed by a measurement receiver as described with reference to FIGS. 6 through 9.

At 1020, the ventilation dashboard server may identify a first display range of the first set of values for the first physiological parameter based on the received first measured value. The operations of 1020 may be performed according to the methods described herein. In some examples, aspects of the operations of 1020 may be performed by a first range component as described with reference to FIGS. 6 through 9.

At 1025, the ventilation dashboard server may identify a second display range of the second set of values for the second physiological parameter based on the received second measured value. The operations of 1025 may be performed according to the methods described herein. In some examples, aspects of the operations of 1025 may be performed by a second range component as described with reference to FIGS. 6 through 9.

At 1030, the ventilation dashboard server may display, in the first row and in the second row, a first visual indicator that highlights the first display range in the first row and the second display range in the second row. The operations of 1030 may be performed according to the methods described herein. In some examples, aspects of the operations of 1030 may be performed by a display component as described with reference to FIGS. 6 through 9.

At 1035, the ventilation dashboard server may display, in the first row and in the second row, a second visual indicator that updates a characteristic of the first visual indicator for the first display range and the second display range based on whether the first display range and the second display range visually overlap between the first row and the second row. The operations of 1035 may be performed according to the methods described herein. In some examples, aspects of the operations of 1035 may be performed by an overlap component as described with reference to FIGS. 6 through 9.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A method for displaying physiological data in a user interface, comprising: identifying a first table from a plurality of tables, wherein the first table defines a ratio between a first set of values for a first physiological parameter and a second set of values for a second physiological parameter; displaying a first row including the first set of values for the first physiological parameter and a second row including the second set of values for the second physiological parameter, wherein the first row and the second row are adjacent and aligned with each other; receiving, from a medical device associated with a patient, a first measured value associated with the first physiological parameter and a second measured value associated with the second physiological parameter; identifying a first display range of the first set of values for the first physiological parameter based at least in part on the received first measured value; identifying a second display range of the second set of values for the second physiological parameter based at least in part on the received second measured value; displaying, in the first row and in the second row, a first visual indicator that highlights the first display range in the first row and the second display range in the second row; and displaying, in the first row and in the second row, a second visual indicator that updates a characteristic of the first visual indicator for the first display range and the second display range based at least in part on whether the first display range and the second display range visually overlap between the first row and the second row.

Aspect 2: The method of aspect 1, wherein displaying the second visual indicator comprises: highlighting the first display range and the second display range a first color based at least in part on the first display range and the second display range overlapping.

Aspect 3: The method of any of aspects 1 through 2, wherein displaying the second visual indicator comprises: highlighting the first display range and the second display range a second color based at least in part on the first display range and the second display range being non-overlapping.

Aspect 4: The method of any of aspects 1 through 3, wherein displaying the first visual indicator comprises: highlighting a subset of the first set of values in the first row that matches the first measured value; and highlighting a subset of the second set of values in the second row that matches the second measured value.

Aspect 5: The method of aspect 4, wherein the subset of the first set of values or the subset of the second set of values comprises duplicated values.

Aspect 6: The method of any of aspects 1 through 5, wherein displaying the first visual indicator comprises: determining that the first measured value is a value between a first value and a second value of the first set of values in the first row; and highlighting the first value and the second value of the first set of values in the first row based at least in part on determining that the first measured value is a value between the first value and the second value of the first set of values.

Aspect 7: The method of any of aspects 1 through 6, wherein displaying the first visual indicator comprises: determining that the second measured value is a value between a first value and a second value of the second set of values in the second row; and highlighting the first value and the second value of the second set of values in the second row based at least in part on determining that the second measured value is a value between the first value and the second value of the second set of values.

Aspect 8: The method of any of aspects 1 through 7, further comprising: receiving a user selection of the first table from the plurality of tables.

Aspect 9: The method of any of aspects 1 through 8, further comprising: displaying, in the user interface, a tile configured to indicate whether the first display range and the second display range overlap based at least in part on the determination.

Aspect 10: The method of any of aspects 1 through 9, wherein the first physiological parameter comprises a fraction of inspired oxygen (FiO2) and the second physiological parameter comprises a positive end-expiratory pressure (PEEP).

Aspect 11: An apparatus for displaying physiological data in a user interface, comprising a processor; memory coupled with the processor; and instructions stored in the memory and executable by the processor to cause the apparatus to perform a method of any of aspects 1 through 10.

Aspect 12: An apparatus for displaying physiological data in a user interface, comprising at least one means for performing a method of any of aspects 1 through 10.

Aspect 13: A non-transitory computer-readable medium storing code for displaying physiological data in a user interface, the code comprising instructions executable by a processor to perform a method of any of aspects 1 through 10.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for displaying physiological data in a user interface, comprising:
identifying a first table from a plurality of tables, wherein the first table defines a ratio between a first set of values for a first physiological parameter and a second set of values for a second physiological parameter;
displaying a first row including the first set of values for the first physiological parameter and a second row including the second set of values for the second physiological parameter, wherein the first row and the second row are adjacent and aligned with each other;
receiving, from a medical device associated with a patient, a first measured value associated with the first physiological parameter and a second measured value associated with the second physiological parameter;
identifying a first display range of the first set of values for the first physiological parameter based at least in part on the received first measured value;
identifying a second display range of the second set of values for the second physiological parameter based at least in part on the received second measured value;
displaying, in the first row and in the second row, a first visual indicator that highlights the first display range in the first row and the second display range in the second row; and
displaying, in the first row and in the second row, a second visual indicator that updates a characteristic of the first visual indicator for the first display range and the second display range based at least in part on whether the first display range and the second display range visually overlap between the first row and the second row.

2. The method of claim 1, wherein displaying the second visual indicator comprises:
highlighting the first display range and the second display range a first color based at least in part on the first display range and the second display range overlapping.

3. The method of claim 1, wherein displaying the second visual indicator comprises:
highlighting the first display range and the second display range a second color based at least in part on the first display range and the second display range being non-overlapping.

4. The method of claim 1, wherein displaying the first visual indicator comprises:
highlighting a subset of the first set of values in the first row that matches the first measured value; and
highlighting a subset of the second set of values in the second row that matches the second measured value.

5. The method of claim 4, wherein the subset of the first set of values or the subset of the second set of values comprises duplicated values.

6. The method of claim 1, wherein displaying the first visual indicator comprises:
determining that the first measured value is a value between a first value and a second value of the first set of values in the first row; and
highlighting the first value and the second value of the first set of values in the first row based at least in part on determining that the first measured value is a value between the first value and the second value of the first set of values.

7. The method of claim 1, wherein displaying the first visual indicator comprises:
determining that the second measured value is a value between a first value and a second value of the second set of values in the second row; and
highlighting the first value and the second value of the second set of values in the second row based at least in part on determining that the second measured value is a value between the first value and the second value of the second set of values.

8. The method of claim 1, further comprising:
receiving a user selection of the first table from the plurality of tables.

9. The method of claim 1, further comprising:
displaying, in the user interface, a tile configured to indicate whether the first display range and the second display range overlap based at least in part on the determination.

10. The method of claim 1, wherein the first physiological parameter comprises a fraction of inspired oxygen (FiO2) and the second physiological parameter comprises a positive end-expiratory pressure (PEEP).

11. An apparatus for displaying physiological data in a user interface, comprising:
a processor,
memory in electronic communication with the processor, and
instructions stored in the memory and executable by the processor to cause the apparatus to:
identify a first table from a plurality of tables, wherein the first table defines a ratio between a first set of values for a first physiological parameter and a second set of values for a second physiological parameter;
display a first row including the first set of values for the first physiological parameter and a second row including the second set of values for the second physiological parameter, wherein the first row and the second row are adjacent and aligned with each other;
receive, from a medical device associated with a patient, a first measured value associated with the first physiological parameter and a second measured value associated with the second physiological parameter;
identify a first display range of the first set of values for the first physiological parameter based at least in part on the received first measured value;
identify a second display range of the second set of values for the second physiological parameter based at least in part on the received second measured value;
display, in the first row and in the second row, a first visual indicator that highlights the first display range in the first row and the second display range in the second row; and
display, in the first row and in the second row, a second visual indicator that updates a characteristic of the first visual indicator for the first display range and the second display range based at least in part on whether the first display range and the second display range visually overlap between the first row and the second row.

12. The apparatus of claim 11, wherein the instructions to display the second visual indicator are executable by the processor to cause the apparatus to:
highlight the first display range and the second display range a first color based at least in part on the first display range and the second display range overlapping.

13. The apparatus of claim 11, wherein the instructions to display the second visual indicator are executable by the processor to cause the apparatus to:
highlight the first display range and the second display range a second color based at least in part on the first display range and the second display range being non-overlapping.

14. The apparatus of claim 11, wherein the instructions to display the first visual indicator are executable by the processor to cause the apparatus to:
highlight a subset of the first set of values in the first row that matches the first measured value; and
highlight a subset of the second set of values in the second row that matches the second measured value.

15. The apparatus of claim 14, wherein the subset of the first set of values or the subset of the second set of values comprises duplicated values.

16. The apparatus of claim 11, wherein the instructions to display the first visual indicator are executable by the processor to cause the apparatus to:
determine that the first measured value is a value between a first value and a second value of the first set of values in the first row; and
highlight the first value and the second value of the first set of values in the first row based at least in part on determining that the first measured value is a value between the first value and the second value of the first set of values.

17. The apparatus of claim 11, wherein the instructions to display the first visual indicator are executable by the processor to cause the apparatus to:
determine that the second measured value is a value between a first value and a second value of the second set of values in the second row; and
highlight the first value and the second value of the second set of values in the second row based at least in part on determining that the second measured value is a value between the first value and the second value of the second set of values.

18. The apparatus of claim 11, wherein the instructions are further executable by the processor to cause the apparatus to:
receive a user selection of the first table from the plurality of tables.

19. The apparatus of claim 11, wherein the instructions are further executable by the processor to cause the apparatus to:
display, in the user interface, a tile configured to indicate whether the first display range and the second display range overlap based at least in part on the determination.

20. The apparatus of claim 11, wherein the first physiological parameter comprises a fraction of inspired oxygen (FiO2) and the second physiological parameter comprises a positive end-expiratory pressure (PEEP).
